(19) United States
(12) Patent Application Publication
(10) Pub. No.: US 2004/0133062 A1
(43) Pub. Date: Jul. 8, 2004

(54) Minimally invasive cardiac force transfer structures

(76) Inventors: Suresh Pai, Mountain View, CA (US); Nicanor Domingo, Brisbane, CA (US); James G. Whayne, Chapel Hill, NC (US)

Correspondence Address: BOZICEVIC, FIELD & FRANCIS LLP, 200 MIDDLEFIELD RD, SUITE 200, MENLO PARK, CA, 94025, US

(21) Appl. No.: 10/682,281

(22) Filed: Oct. 8, 2003

Related U.S. Application Data
(60) Provisional application No. 60/418,018, filed on Oct. 11, 2002.

(51) Int. Cl. A61M 1/12

(52) U.S. Cl. 600/16; 600/37; 601/153; 623/3.21

(57) ABSTRACT

Devices and methods for treating degenerative, congestive heart disease and related dysfunction are described. Minimally invasive surgical force transfer structures offer devices that mitigate changes in the ventricular structure (i.e. remodeling) and deterioration of global left ventricular performance related to tissue damage precipitating from ischemia, acute myocardial infarction (AMI) or other abnormalities. These force transfer structures resist diastolic filling pressure while simultaneously transmitting a compressive force to the aorta, the pulmonary artery, the atrium and/or other anatomic structure to improve cardiac output thereby reducing the strain on the heart. In addition, the force transfer structures may compensate or provide therapeutic treatment for congestive heart failure and/or reverse the remodeling that produces an enlarged heart. The force transfer structures are implanted in target heart regions using less invasive surgical techniques involving port access or small incisions into the thoracic cavity to provide a potential, palliative or therapeutic response to the disease.

### FIELD OF THE INVENTION

The present invention relates generally to minimally invasive, mechanical, medical devices for treating or preventing congestive heart failure and related or concomitant vascular dysfunction. More specifically, the invention relates to force transfer structures that mitigate changes in the ventricular and/or atrial structure and geometry and deterioration of global left and right ventricular and atrial performance related to tissue damage from myocardial ischemia, acute myocardial infarction (AMI), valve related disease or dysfunction, vascular related dysfunction, or other instigators of deterioration of cardiac output and/or function.

### BACKGROUND

Congestive heart failure (CHF) is a progressive and lethal disease if left untreated. The CHF syndrome often evolves as a continuum of clinical adaptations, from the subtle loss of normal function to the presence of symptoms refractory to medical therapy. While the exact etiology of the syndrome that causes heart failure is not fully understood, the primary cause of CHF is the inability of the heart to properly and adequately fill or empty blood from the left ventricle (i.e. left ventricular dysfunction) with adequate efficiency to meet the metabolic needs of the body.

In addition, non-cardiac factors can also be activated due the overall degenerative cycle that ensues. These include neuro-hormonal stimulation, endothelial dysfunction, vasoconstriction, and renal sodium retention all of which can cause dyspnea, fatigue and edema rendering patients unable to perform the simplest everyday tasks. These types of non-cardiac factors are secondary to the negative, functional adaptations of the ventricles, cardiac valves or load conditions applied to or resisted by these structures. Even with novel pharmacological, surgical and device-based therapies, symptoms can be alleviated, but the quality of life remains significantly impaired and the associated morbidity and mortality of the disease is exceptionally high.

Ischemic heart disease is currently the leading cause of CHF in the western world, accounting for greater than 70% of cases worldwide. In these cases, CHF can precipitate from ischemic conditions or from muscle damage (i.e. AMI due to obstruction of a coronary artery) which can weaken the heart muscle, initiating a process known as remodeling where changes in cardiac anatomy and physiology include ventricular dilatation, regional wall motion abnormalities, decreases in the left ventricular ejection fraction and impairment of other critical parameters of ventricular function. This left ventricular dysfunction is further aggravated by hypertension and valvular disease in which a chronic volume or pressure overload can alter the structure and function of the ventricle. Decreases in systolic contraction can lead to cardiomyopathy, which further exacerbates the localized, ischemia damaged tissue or AMI insult into a global impairment leading to episodes of arrhythmia, progressive pump failure and death.

Analogous to aneurysms in diseased hearts accompanying abnormally thin and weak myocardial tissue, ischemia-damaged and/or infarct damaged heart muscle tissue results in progressive softening or degeneration of cardiac tissue. These ischemic and infarcted zones of the heart muscle wall have limited, if not complete loss of tissue contractile functionality and overall physical integrity. Also, the disease is usually associated with a progressive enlargement of the heart as it increases contractility and heart rate in a compensatory response to the decreasing cardiac output. With this enlargement, the heart's burden is increased to pump more blood with each pump cycle. A phenomenon known as myocardial stretch is implicated in the perpetual cyclic feedback loop that causes areas of compromised heart muscle tissue to bulge outward. When the bulging is related to AMI, this behavior is characterized as infarct expansion. With this bulging, the heart's natural contraction mechanism is dissipated into and attenuated resulting in a marked and progressing decrease in cardiac output.

Normal cardiac valve closure (especially that of the mitral valve) is dependent upon the integrity of the myocardium, as well as that of the valve apparatus itself. The normal mitral valve is a complex structure; consisting of leaflets, annulus, chordae tendineae, and papillary muscles and any damage or impairment in function of any of these key components can render a valve structure incompetent. Impairment of valve function, due to independent factors (i.e. a concomitant valve pathology) or dependent factors (i.e. valve dilation related to dilated cardiomyopathy or mitral regurgitation due to atrial enlargement), can result in valvular insufficiency further exacerbating the degenerative CHF cycle.

The major objectives of heart failure therapy are to decrease symptoms and prolong life. The American Heart Association guidelines suggest that the optimal treatment objectives includes means to increase survival, exercise capacity, improve of quality of life, while decreasing symptoms, morbidity and the continued progression of the degeneration. Various pharmacological and surgical methods have been applied both with palliative and therapeutic outcome goals, however there still remains no cure for the condition.

Modern pharmacological approaches such as diuretics, vasodilators, and digoxin dramatically lessen CHF symptoms and prolong life by mitigating the non-cardiac factors implicated in the syndrome. Furosemide (more commonly known as Lasix) is also a valuable diuretic drug which eliminates excess water and salt from the body by altering kidney function and thereby increasing urine output thereby relieving the circulatory congestion and the accompanying pulmonary and peripheral edema. Vasodilators, like angiotensin-converting-enzyme (ACE) inhibitors have become one of the cornerstones in treatment of heart failure. These kinds of vasodilators relax both arterial and venous smooth muscle, thereby reducing the resistance to left ventricular ejection. In patients with enlarged ventricles, the drug increases stroke volume with a reduction in ventricular filling pressure. Digoxin has also been found to be positively inotropic (i.e. strengthens the heart's contraction capability).

On the surgical front, cardiomyoplasty is a recently developed treatment of CHF, where the latissimus dorsi muscle is removed from the patient's shoulder, wrapped around the heart and chronically paced in synchrony with ventricular systole with the goal of assisting the heart to pump during systole. The procedure is known to provide some symptomatic improvement, but is controversial with regard to its ability to enable active improvement of cardiac performance. It is hypothesized that the symptomatic improvement is primarily generated by passive constraint and mitigation of the degenerative, remodeling process. In spite of the positive outcome on relieving some of the symptoms, the procedure is highly invasive, requiring access to the heart via a stemotomy, expensive, complex and of unknown durability (due to the muscle wrap blood flow requirements and fibrosis issues). Another surgery of interest is an innovative procedure developed by R. Bautista, MD. In this procedure, the overall mass, volume and diameter of the heart are physically reduced by dissection and removal of left ventricular tissue. Besides being a highly invasive, traumatic and costly procedure, the actual volume reduction results in a reduction in valve competence and elicits the associated regurgitation. Surgical treatment of valvular dysfunction also includes a wide range of open procedure options ranging from mitral ring annuloplasty to complete valve replacement using mechanical or tissue based valve prosthesis. While being generally successful and routine in surgical practice today, these procedures are also costly, highly invasive and are still have significant associated morbidity and mortality.

More recently, mechanical assist devices which act as a bridge to transplant such as the left ventricular assist device (LVAD) or the total artificial heart (TAH) implant have become available. LVAD's are implantable, mechanical pumps that facilitate the flow of blood from the left ventricle into the aorta. The latest, TAH technologies feature many improved design and material enhancements that increase their durability and reliability. However, the use of such devices is still limited by high costs and a lack of substantial, clinical evidence warranting their use.

Other device-based options for this patient subset include reshaping, reinforcement and reduction of the heart's anatomical structure using polymeric and metallic bands, cuffs, jackets, balloon/balloon-like structures or socks to provide external stress relief to the heart and to reduce the propensity/capability of the cardiac tissue to distend or become continually stretched and damaged with progressive pump cycles. Examples of such devices are U.S. patent Publication No. 2002/0045799 and U.S. Pat. No. 5,702,343. In addition, devices are being studied that attempt to prevent the tissue remodeling using tethers and growth limiting struts or structures described in various patents (e.g., U.S. Pat. No. 6,406,420). In general, all of these concepts support the cardiac muscle and restrict growth externally and globally via surgical placement about the epicardium and in some instances are positioned across the cardiac muscle tissue. As such, these types of approaches require unnecessary positioning of the devices over healthy (non local, undamaged) areas or zones of the heart affecting the entire organ when the primary treatment is usually focused is on the left ventricle or the mitral valve annulus. This non-localized treatment can elicit iatrogenic conditions such as undesired valvular dysfunction or constrictive physiology due to over restriction of the heart by such restraints.

Recently, several device based options have also been introduced where implants are positioned by minimally invasive means in the coronary sinus in one configuration and then assume a post deployment configuration that constricts around the annulus to improve valve competence in dilated cardiomyopathy (e.g., U.S. patent Application No. 2002/016628). The clinical efficacy of this approach while appealing is unknown at this time.

Finally, the ultimate treatment for people suffering end stage CHF is a heart transplant. Transplants represent a massive challenge with donor hearts generally in short supply and with the transplant surgery itself presenting a high risk, traumatic and costly procedure. In spite of this, transplants present a valuable, albeit limited, upside increasing life expectancy of end stage congestive heart failure patient from less than one year up to a potential five years.

It is evident that there is currently no ideal treatment among the various surgical, pharmacological, and device based approaches to treat the multiple cardiac and non-cardiac factors implicated with the syndrome of CHF. There is a clear, unmet clinical need for technology that is minimally invasive (ideally percutaneous) which can prevent, treat or reduce the structural remodeling to the heart and it's sub-structures across the continuum of the syndrome beginning acutely with the ischemia or ischemic infarct through the end stages where there is often left ventricular and valvular dysfunction refractory to conventional treatments.

Accordingly, there is a need for a force transfer structure that can be utilized to improve cardiac output, reduce wall stresses, reinforce the walls, and reduce/limit volume of the heart muscle as required using percutaneous, minimally invasive surgical (MIS), and open surgical means or a combination thereof. Ideally such a device could facilitate operator controlled “tailoring” of treatment using various embodiments of the invention at various chosen target zones (i.e. ventricles, atria, aorta, pulmonary artery, etc.). The custom tailoring of each force transfer structure also enables application of compressive forces against a specific anatomy in a direct resultant response (i.e. energy transfer) to action of another anatomy and patient specific adjustability of the amount of compression applied to the anatomy to optimize the heart's overall hemodynamic performance.

Patients suffering from severe CHF, who are unresponsive to medication, are generally precluded to open surgical approaches and potentially awaiting transplant could derive massive and direct benefit from a minimally invasive device to limit further degeneration of their condition. In addition, the implant embodiments of the invention can also facilitate positive or reverse remodeling (i.e. to provide a mild compressive force against the dilated ventricle and transfer compressive forces to other anatomic structures to induce pulsatile contraction of these structures to facilitate improved cardiac output and efficiency).

### SUMMARY OF THE INVENTION

The embodiments of the present invention describe force transfer structures that direct contraction and expansion forces of the heart's ventricles to invoke a pulsatile motion of other anatomic structures such as the atria, the aorta, the pulmonary artery, or other anatomy to influence and modify cardiac output. That is, the embodiments allow the contractile and expansion energies of the heart to be transferred to the atria, aorta, the pulmonary artery, or other anatomy improving the efficiency of pumping thereby improving cardiac output. Another potential benefit of these force transfer structures is that they may also work in concert to provide reinforcement against myocardial stretch (or infarct expansion). Especially in this regard, the present invention is advantageously employed in connection with the featured described in U.S. patent application Atty. Docket No. EXMA-001, entitled “System for Heart Treatment,” filed on even date herewith that claims benefit to U.S. Provisional Application Serial No. 60/329,694, entitled “Percutaneous Cardiac Support Structures and Deployment Means,” filed Oct. 16, 2001 and Provisional Application Serial No. 60/368,918 entitled “Percutaneous Vascular Tensioning Devices and Methods,” filed Mar. 29, 2002—each document being incorporated herein in its entirety by reference.

Whether provided in a system with such ancillary equipment/devices or not, devices and approaches as taught herein provide dynamic support or reinforcement that is active throughout the cardiac cycle unlike previous device approaches that solely reduce the stress in the heart wall during diastole. Diastolic compliance can also be regulated or controlled with such structures.

The force transfer structures of the present invention can be implanted/deployed using a minimally invasive, surgical approach. In practice, such structures can be used to produce an induced contraction of the atria in response to dilation of the ventricles and allow expansion of the atria during contraction of the ventricles. Similarly, the force transfer structures can induce contraction of the aorta, pulmonary artery, or other vascular conduit during diastole and allow expansion during systole. As such, these affected alternate anatomic structures apart from the ventricles, are assisting in aiding in the systemic pumping of blood throughout the cardiac cycle relieving the heart of overburden for this role and also improving cardiac output.

### BRIEF DESCRIPTION OF THE DRAWINGS

The following drawings and associated description describe aspects of the present invention. Neither is to be taken in a limiting sense, but is made merely for the purpose of illustrating the general principles of the inventions. Certain aspects of the figures diagrammatically represent the present invention, while others may be indicative of preferred relations. Regardless, variation of the invention from what is shown in the figures is contemplated.

FIGS. 1A and 1B show perspective views dramatizing a healthy heart in systole and diastole respectively.

FIGS. 2A and 2B show perspective views dramatizing a diseased (enlarged) heart in systole and diastole respectively.

FIGS. 3A and 3B show a top view and a perspective view of a force transfer structure embodiment; FIGS. 3C and 3D show a top view and a perspective view of an alternative force transfer structure embodiment.

FIGS. 4A and 4B show perspective views of a heart with two force transfer structure embodiments secured to the aorta and the ventricles to impart motion in the aorta in response to beating (contraction/expansion) of the heart.

FIG. 5A shows an alternative force transfer structure embodiment; FIG. 5B shows the force transfer structure embodiment in FIG. 5A secured about the ventricle and aorta of a heart.

FIGS. 6A to 6G show a top view, three cross-sectional views, a perspective view, a side view, and a side-sectional view of a force transfer structure embodiment incorporating a fluid displacement mechanism to transmit compressive forces from the ventricle to another anatomic structure.

FIG. 7 shows an alternative force transfer structure embodiment incorporating a fluid displacement mechanism.

FIGS. 8A and 8B show two fluid transmission tube embodiments that allow fluid transport between various regions of the force transfer structure embodiments in FIGS. 6A to 6G, and 7.

FIGS. 9A and 9B show perspective views of the force transfer structure embodiment in FIGS. 6A to 6G attached to an aorta and a heart during systole and diastole respectively.

FIGS. 10A and 10B show side views of the force transfer structure embodiment in FIG. 7 attached to an aorta and a heart during systole and diastole respectively.

FIG. 11 shows a perspective view of an alternative force transfer structure embodiment secured to the aorta and about the ventricles.

FIG. 12 shows a perspective view of a force transfer structure embodiment secured about the atria and the ventricles.

FIG. 13 shows a perspective view of a force transfer structure embodiment secured to the pulmonary artery and about the ventricles.

FIG. 14 shows a perspective view of a force transfer structure embodiment secured about the atria, the aorta, and the pulmonary artery, and to the ventricles.

FIG. 15 shows a perspective view of a force transfer structure embodiment secured about the aorta and the pulmonary artery, and to the ventricles.

### DETAILED DESCRIPTION

Having described the characteristics and problems of congestive heart disease, the treatment method and apparatus of the present invention will now be described. The embodiments of the invention described below provide a solution to treating congestive heart disease, and the contributing or associated co-morbid, anatomical, and physiological deficiencies. Addressing the multiple factors that affect or cause congestive heart disease can retard or reverse the implicated remodeling thereby treating or mitigating the congestive heart disease and associated symptoms.

Before the present invention is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “and,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs.

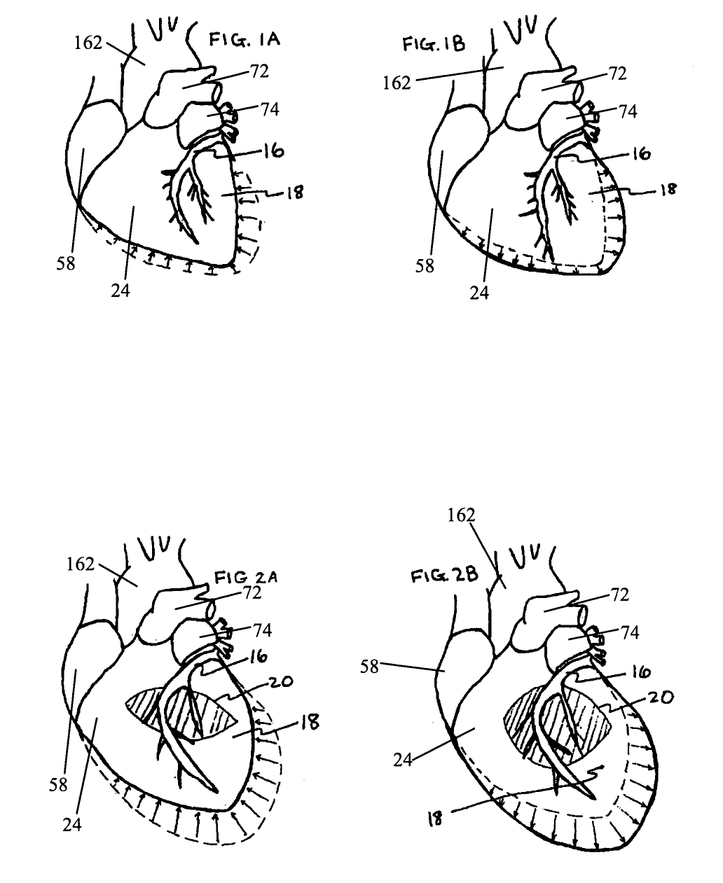

Turning now to FIGS. 1A and 1B, an anterior view of a healthy heart in systole and diastole respectively is shown with directional arrows showing motion of the heart. During normal motion and function of the heart, the aorta 162 being a compliant vessel responds and pulsates to aid in pumping blood throughout the body. The aorta actually expands to increase the volume of blood stored by the aorta 162 as the left ventricle 18 and right ventricle 24 contract during systole. Upon closure of the aortic valve and the subsequent expansion of the ventricles during diastole, the aorta contracts and continues to push blood throughout the body. Similarly, the pulmonary artery 72 expands to store blood during systole and helps continue to pump blood into the lungs during diastole. The left atrium 74 expands to accept blood from the lungs during systole and contracts during diastole to help pump blood into the left ventricle 18. The right atrium 58 expands to accept blood from the vena cava during systole and contracts during diastole to help pump blood into the right ventricle 24.

In FIGS. 2A and 2B, perspective views are shown of a diseased (enlarged) heart in systole and diastole respectively. The infarcted or ischemic region 20 is shown to stretch from systole to diastole consistent with the progressive remodeling that occurs due to increased diastolic filling pressures exerted on the diseased tissue. A radial and axial expansion that is experienced by the heart leads to degenerative remodeling and concomitant organ enlargement. This enlargement can be localized along the anterior wall of the left ventricle 18, can be located or extend septally, can include the right ventricle 24, and/or can involve the mitral valve annulus. This remodeling is exacerbated and accelerated by deterioration of associated anatomic structures such as the atria 74 and 58, the aorta 162, the pulmonary artery 72, etc. which aid cardiac output in normal hearts by pulsating and augmenting the pumping action of the heart ventricles alone.

Force Transfer Structures with Tensile Members

The force transfer structures of the invention regulate diastolic filling of the ventricles while transmitting ventricular wall motion to induce an improved expansion/contraction motion and response of the aorta 162, the right and left atria 58 and 74, the pulmonary artery 72, and/or other anatomic structure by initiating and coordinating the pumping motion of these structures in response to motion of the ventricles thereby improving the pumping efficiency of the heart.

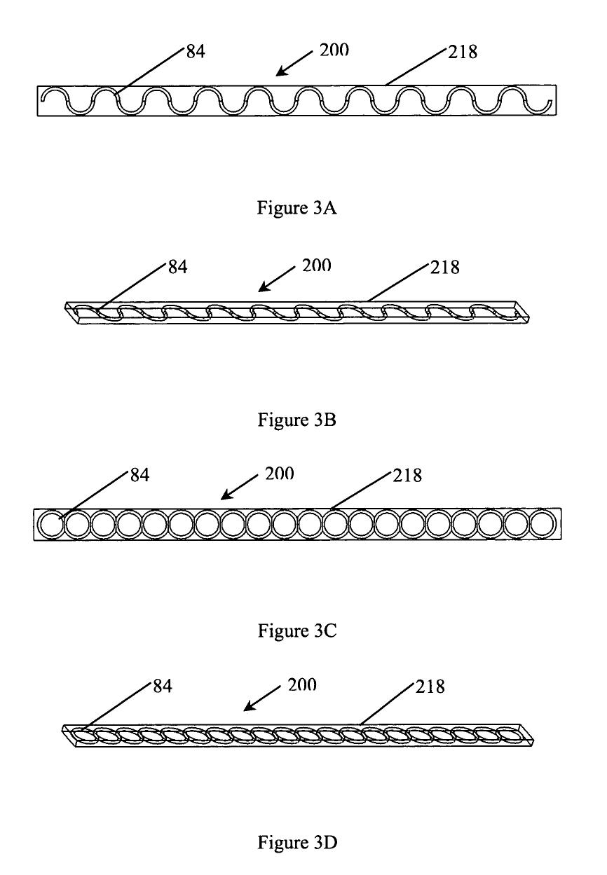

FIGS. 3A and 3B show a top view and a perspective view of a force transfer structure 200 embodiment that incorporates a tensile member 84 embedded in a polymeric covering 218. The tensile member 84 in this embodiment comprises a metal, alloy (e.g., superelastic material such as nickel titanium), superelastic polymer, or other material fabricated into a wire, flat ribbon, or other geometry, and thermally formed into a sinusoidal pattern. The tensile member 84 is embedded between layers of a covering 218 selected from expanded PTFE, polyester, nylon, silicone, urethane, derivatives of these materials, composite materials or other polymers. The covering 218 can be injection molded, extruded, adhesively bonded, ultrasonically welded, thermally bonded, radio frequency welded, or secured using another methodology to encompass or secure to the tensile member 84.

FIGS. 3C and 3D show a top view and a perspective view of an alternative force transfer structure 200 embodiment that incorporates a tensile member 84 comprising a flat ribbon or sheet raw material that is laser cut, chemically etched, milled, or otherwise cut into a pattern of circular cells linked together. The tensile member 84 is embedded into a covering 218 as described above. The tensile member 84 can alternatively comprise any pattern cut into a 2-dimensional sheet or flat ribbon raw material, a tube cut into a 3-dimensional pattern, or a sheet or flat ribbon cut into a pattern and formed into a 3-dimensional structure.

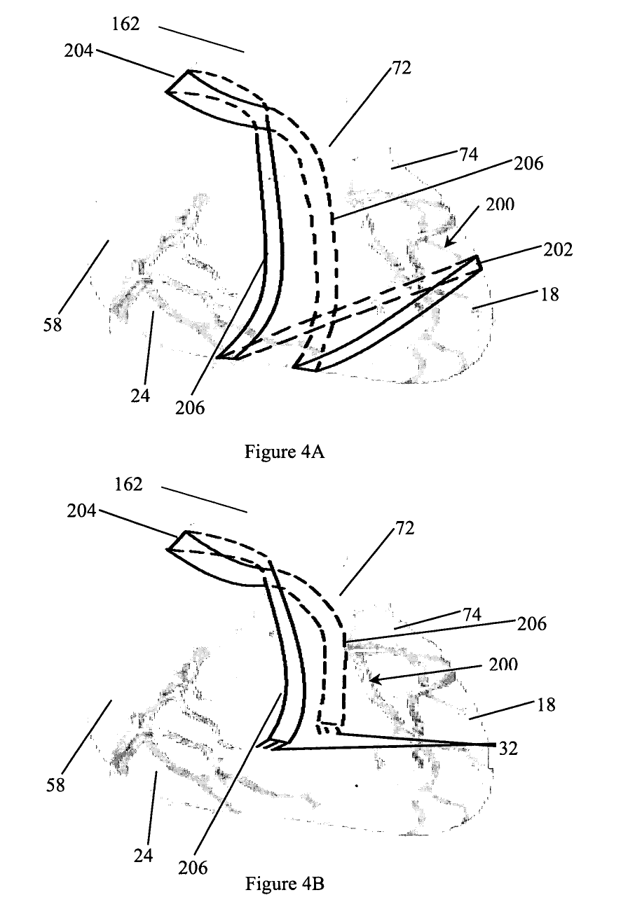

FIG. 4A shows a force transfer structure 200, such as those shown in FIGS. 3A to 3D, secured about heart ventricles along a source loop 202 and in this illustration attached about the aorta 162 along a recipient loop 204. The dotted lines show the path for the force transfer structure 200 along the posterior side of the heart and the solid lines show the force transfer structure along the anterior side of the heart. As shown in FIG. 4A, a single force transfer structure having sufficient column strength and elasticity to open the recipient loop 204 as the source loop 202 is relaxed and compress the recipient loop 204 as the source loop 202 is expanded is wrapped around the ventricles and the aorta. A force transmission link 206 connects the source loop 202 and the recipient loop 204 and induces the motion of the recipient loop 204 in response to movement in the source loop 202. As such, the aorta 162 can be induced to contract and expand along with the contraction and expansion of the left and right ventricles 18 and 24. As the left and right ventricles 18 and 24 expand during diastolic filling, the source loop 202 correspondingly enlarges causing the recipient loop 204 to contract thereby externally compressing the covered portion of the aorta 162 (or pulmonary artery, atria, or other anatomic structure not shown) around which the recipient loop 204 is wound. As the left and right ventricles 18 and 24 contract during systole, the source loop 202 relaxes and returns towards its preformed shape (for superelastic and elastic materials) thereby allowing the recipient loop 204 to relax into an enlarged shape allowing the aorta to expand to its resting or equilibrium condition. Alternatively, the force transfer structure can be affixed to the ventricles and aorta and incorporate a column strength in the tensile member 84 to actively expand the recipient loop 204 and thereby the aorta 162 as the source loop 202 is compressed coincident with the contraction of the left and right ventricles 18 and 24.

The ends of the force transfer structure 200 in FIG. 4A are secured together to comprise a complete wrapped structure. The free ends of the force transfer structure can incorporate ties that can be knotted together thereby defining the inherent compression placed upon the heart. Alternatively, a zip-tie ratcheting mechanism or a twist-tie feature, or similar interface, can be incorporated at the free ends to provide rapid attachment of the force transfer structure 200 and/or enable tightening the force transfer structure over time. Other attachment means can be utilized to secure free ends of the force transfer structure including adhesives, staples, ultrasonic welding, thermal bonding, suturing directly or to a metallic or polymeric cuff, combinations of these various means or other modality.

As shown in FIG. 4B, the force transfer structure 200 can be wound around the aorta 162 at the recipient loop 204 and have the free ends of the structure secured to opposite sides of the left ventricle 18 using anchor formations 32. As previously stated, the dotted lines represent the structure placed along the posterior surface of the heart and the solid lines show the structure along the anterior surface. The anchor formations 32 consist of staples, barbs designed to penetrate into or through the myocardium, suture stitches, adhesive drops, or other attachment means capable of securing the force transfer structure and maintain the bond and intimate tissue contact when the implant is exposed to the anticipated stress conditions associated with applying force to the recipient loop 204 as the left ventricle 18 expands and contracts.

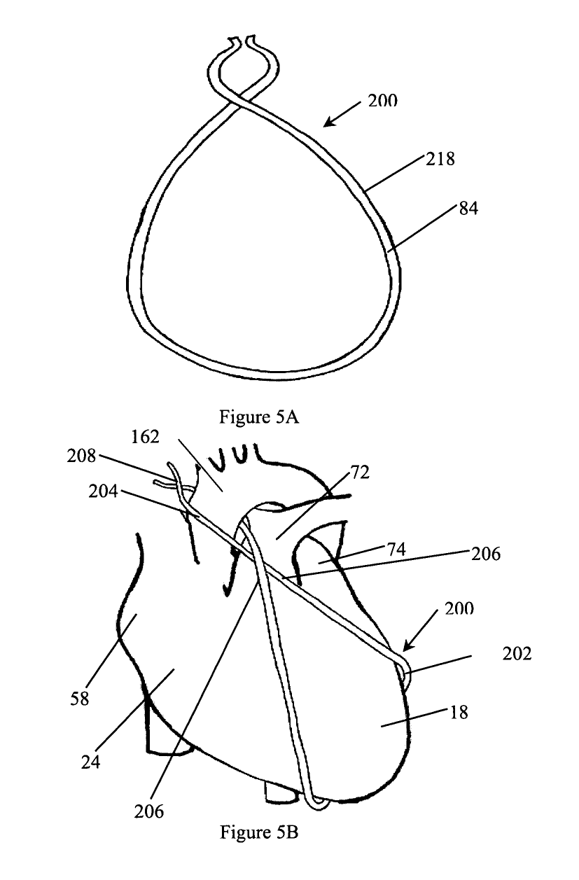

FIG. 5A shows an alternative force transfer structure 200 embodiment comprising a wire or flat ribbon tensile member 84 fabricated from a metal or biocompatible alloy formed into a source loop 202 and a recipient loop 204 with the force transmission link 206 consisting of an intersection of the tensile member 84. The tensile member 84 of the force transfer structure 200 can be embedded in a covering 218. As shown in FIG. 5B, the force transfer structure 200 is wound around the left and right ventricles 18 and 24 at a source loop 202 and around the aorta 162 at the recipient loop 204. The free ends of the force transfer structure 200 are secured together at a connection 208 such that the desired transmission of motion and energy into the aorta in response to movement of the ventricles is obtained. The connection 208 can be produced by twisting the free ends of the force transfer structure 200 (especially if the tensile member is malleable) thereby tying the ends together, knotting the free ends, using another suture, staple, clip, suture, combination of these means or other attachment means to secure the free ends, or other mechanism.

These force transfer structures 200 can be secured around the aorta 162 and ventricles 18 and 24 taut so any motion of the heart causes immediate and significant pulsation (or radial compression) of the aorta 162, or loosely to produce a delay or reduction in the amount of motion transmitted from the heart to the aorta 162. The ratio between the diameter of the recipient loop 204 extending around the aorta 162 and the separation between the anchor formations 32 or the diameter of the source loop 202 of the force transfer structure 200 can define the desired difference in contraction and expansion ratios between the left and right ventricles 18 and 24 and the aorta 162. As the left and right ventricles 18 and 24 expand during diastole, tension is applied to the force transfer structure transmitting a reactive force to the recipient loop 204 causing the aorta 162 to simultaneously contract producing a pulsatile motion of the aorta that improves the cardiac output. During systole, as the left and right ventricles 18 and 24 contract, the aorta 162 is allowed to or urged to expand producing the desired filling of the aorta 162.

These force transfer structure 200 embodiments increase cardiac output by utilizing the aorta 162 as a reservoir capable of cycling with the heart and facilitating the pumping of blood throughout the vasculature. In a similar way, the recipient loop 204 of the force transfer structure 200 can alternatively be secured about the pulmonary artery 72, right and left atria 58 and 74, or other anatomic structure to induce pulsatile motion of these anatomic structures relative to and directed by motion of the left and right ventricles 18 and 24 thereby improving cardiac output in synchrony of blood pumped with the normal cardiac cycle.

Force Transfer Structures Utilizing Fluid Transmission

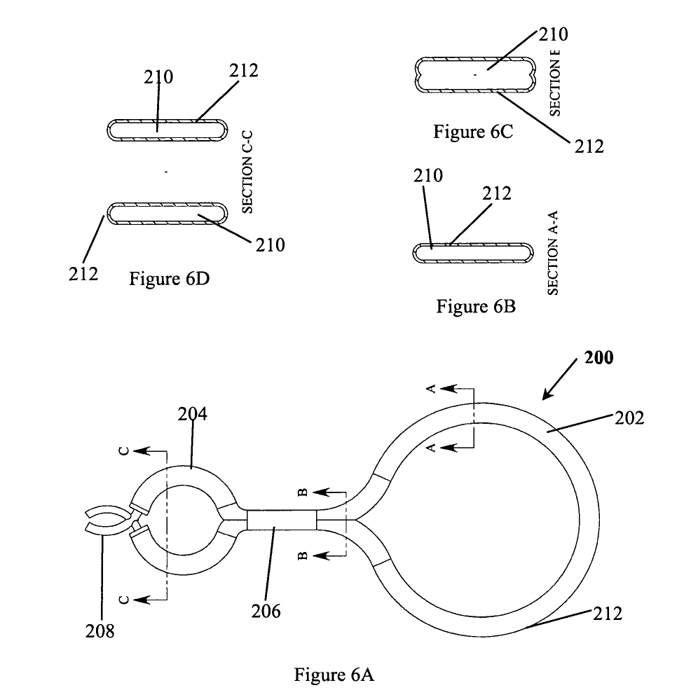

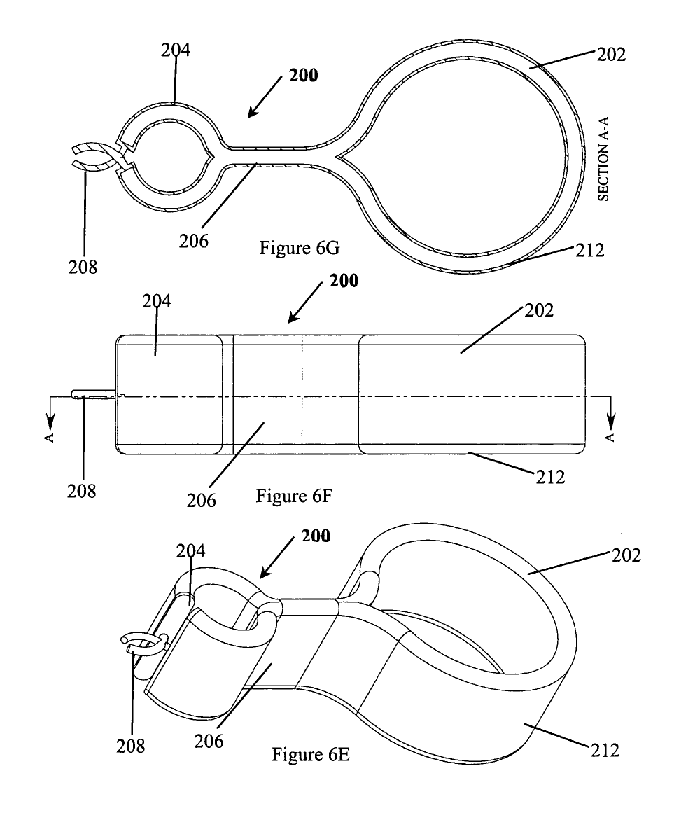

FIG. 6A shows a top view of a force transfer structure that incorporates a fluid transmission mechanism to impart motion of the aorta, pulmonary artery, atria, or other anatomic structure contacting a recipient loop 204 by motion of the left and right ventricles 18 and 24 contacting a source loop 202. Unlike the force transfer structure 200 embodiments incorporating tensile members 84 described above that must move along the tissue surface to compress and/or expand the aorta, pulmonary artery, atria, or other anatomic structure in response to expansion and/or contraction of the ventricles, the fluid transmission embodiments described below induce motion in these various anatomic locations by transferring fluid between chambers located at the source loop 202 and the recipient loop 204. As such, the fluid transmission force transfer structure embodiments minimize the potential of abrading the tissue surface.

The force transfer structure 200 in FIG. 6A incorporates a source loop 202 that contains a wall 212 defining a first bladder or chamber, a recipient loop 204 that contains a wall 212 defining a second bladder or chamber, a transmission link 206 that contains a lumen that feeds the source loop 202 and the recipient loop 204, and connection means 208 to tie or otherwise secure opposite ends of the recipient loop 204 of the force transfer structure 200. A similar connection means 208 to tie or otherwise secure opposite ends of the source loop can also be configured (not shown). FIG. 6B shows a cross-section of the source loop 202 of the force transfer structure 200 in FIG. 6A taken along section A-A. The wall 212 of the chamber defines a lumen 210 into which fluid resides and flows. The ovalized cross-section of the source loop 202 enables contacting a wider region of and more intimate contact with the left and right ventricles 18 and 24 facilitating better distribution of the forces applied to the source loop 202. Similarly, the recipient loop 204 has an ovalized cross-section, as shown in FIG. 6D which is taken along C-C. FIG. 6C shows the cross-section along B-B at the intersection between the source loop 202 and the transmission link 206.

FIGS. 6E to 6G show a perspective, side, and side-sectional view taken along A-A of FIG. 6F. As shown in FIG. 6G, the lumen 210 defined by the wall 212 of the force transfer structure 200 routes from the source loop 202 through the transmission link 206 and ending at the opposite ends of the recipient loop 204. The source loop 202 defines an enclosed loop to fit over the apex of the heart about the right and left. The recipient loop 204 provides free ends that can be secured about anatomical structures like the aorta, pulmonary artery, atria, or others at a connection 208 by tying, stapling, zip-tying, twist tying, suturing, combinations of these various means, or other means for attaching the free ends together.

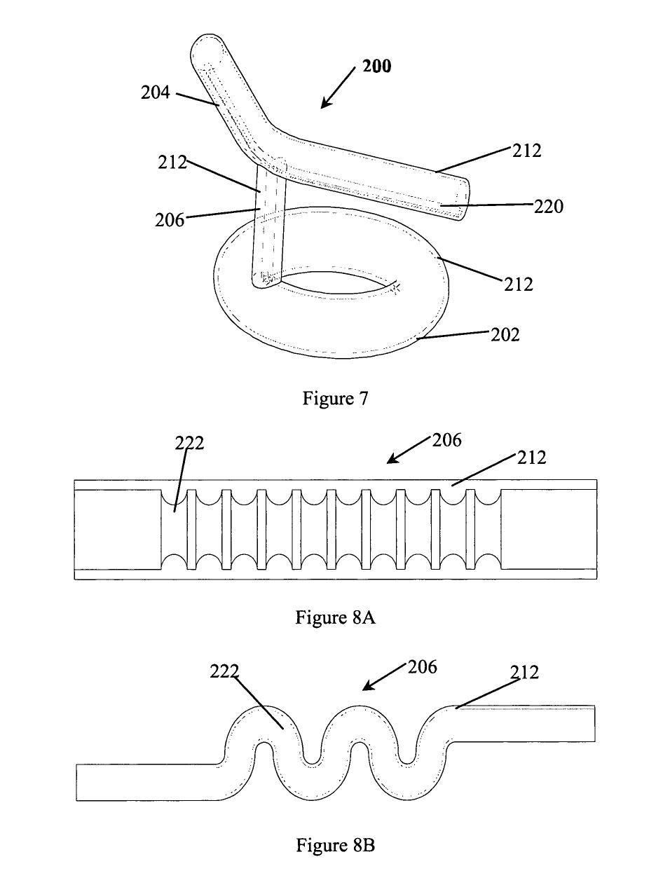

As shown in FIG. 7, the transmission link 206 of the force transfer structure 200 can comprise a different cross-section geometry than the recipient loop 204 or the source loop 202, unlike that shown in FIGS. 6A to 6G. As shown in FIG. 7, the transmission link 206 comprises an ovalized, circular, or flattened cross-section oriented 180 degrees from the source loop 202 and the recipient loop 204 so that the transmission link 206 lies along the ventricle when the source loop 202 and the recipient loop 204 are secured. This transmission link 206 also incorporates a thicker wall 212 than the source loop 202 and the recipient loop 204 to ensure the effects of the fluid transmission is directed solely and amplified between the source loop 202 and the recipient loop 204. It should also be noted that the wall thickness of the source loop 202 and recipient loop 204 can vary to control the expansion ratio.

The force transfer structure embodiment in FIG. 7 shows additional features such as a malleable or superelastic tensile member 220 incorporated along one side of the recipient loop 204 wall 212. This tensile member 220 provides additional support and structure to the recipient loop 204 while also providing a mechanism (other than the connection means 208 in FIGS. 6A to 6G) to secure the recipient loop 204 around the aorta, pulmonary artery, atria, or other anatomic structure. It should be noted that one or more tensile members 220 can be incorporated along the transmission link 206 and/or the source loop 202.

FIGS. 8A and 8B show two alternative transmission link 206 embodiments that incorporate a flow limiter/controller to stabilize the force transfer structure, increase the flexibility of the transmission link 206, and/or provide a fluid resistor between the source and recipient loops. Such a fluid resistor is capable of not only reducing/moderating the amount of force transmitted between the loops, but alsophase shifting the timing of the force transmitted from the source loop 202 to the recipient loop 204. Various fluid resistor designs of which FIGS. 8A and 8B are representative examples could be optimized to provide energy transfer to enable pumping in synchrony with the tempo and pattern of the normal cardiac cycle.

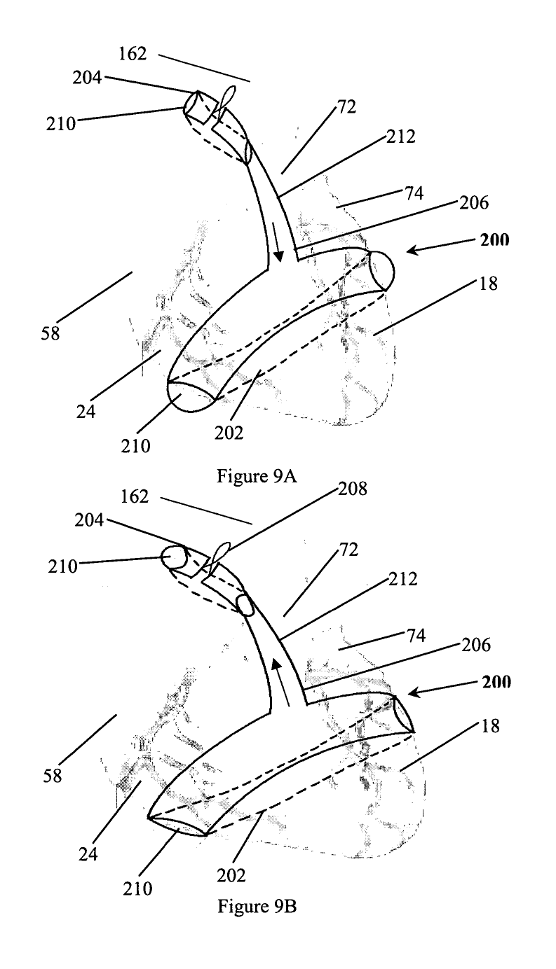

FIGS. 9A and 9B show perspective views of the force transfer structure embodiment in FIGS. 6A to 6G with the source loop 202 secured about the left and right ventricles 18 and 24, and the recipient loop 204 secured at a connector 208 about the aorta 162. FIG. 9A shows the force transfer structure position and shape during systole when the ventricles have maximally contracted. In this stage of the cardiac cycle, the contracted ventricles produce a vacuum in the source loop 202 causing fluid to flow through the transmission link 206 from the recipient loop 204 to the source loop 202 thereby allowing or encouraging the aorta 162 to enlarge. FIG. 9B shows the force transfer structure position and shape during diastole when the ventricles have maximally expanded. In this stage of the cardiac cycle, the expanded ventricles have stretch or extend the source loop 202 and expel fluid through the transmission link 206 and into the recipient loop 204 which expands causing the aorta to contract in reaction to the resultant, compressive force. The amount of fluid (e.g. saline, water, CO₂, or other such biocompatible fluid) within the force transfer structure 200 affects the force applied or transferred from the source loop 202 to the recipient loop 204 and also influences the amount of reinforcement against myocardial stretch or infarct expansion the source loop 202 exerts against the ventricles.

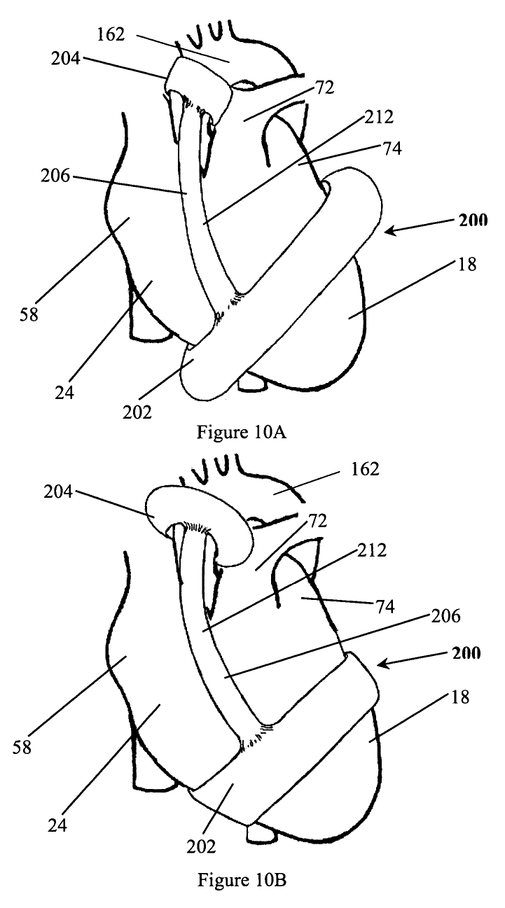

FIGS. 10A and 10B show side views of the force transfer structure in FIG. 7 during systole and diastole respectively. As in previous embodiments, when the left and right ventricles 18 and 24 contract during systole, the aorta 162 is allowed or urged to expand and when the ventricles expand during diastole, the aorta is forced to contract.

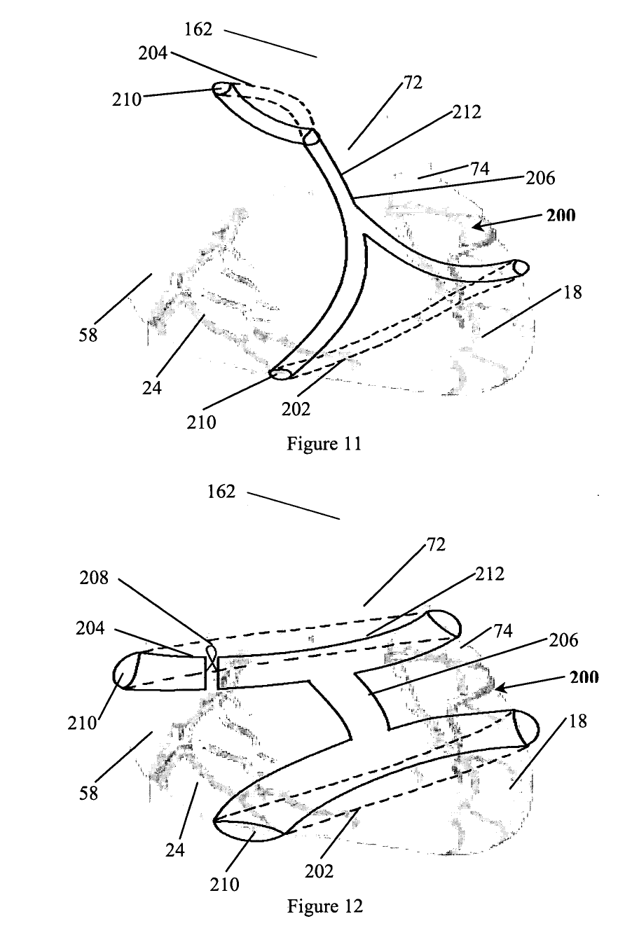

FIG. 11 shows an alternative force transfer structure 200 embodiment that comprises a source loop 202 secured around the left and right ventricles 18 and 24, a transmission link 206, and a recipient loop 204 secured around the aorta 162. This embodiment would facilitate placement of the force transfer structure about a lesser portion of the left and right ventricles while still providing the necessary coverage required for adequate loop to recipient loop energy transfer without potential constriction of critical vessels at the level of the source loop 202. The attachment of the source loop 202 could be configured as shown with the Y-junction positioned loosely over and not intimately attached over critical vessels like the left anterior descending artery.

FIG. 12 shows the force transfer structure embodiments in FIGS. 6A to 6G or FIG. 7 secured to the heart with the source loop 202 about the left and right ventricles 18 and 24, and the recipient loop 204 extending around the right atrium 58 and the left atrium 74 such that motion of the left and right ventricles induces motion of the right and/or left atria. It should be noted that the recipient loop 204 can be placed around both atria or just the left or right atrium individually. For individual atrium usage, the recipient loop 204 could be alternatively be fashioned such that the bladder portion of the loops is limited to only a portion of the loop with the remainder of the loop a simple, solid band of material (not shown). This bladder section could then be located over the desired individual atrium For example, by securing the recipient loop 204 around at least the left atrium, diastolic expansion of the left and right ventricles 18 and 24 produces a contraction of the atria causing the left atrium 74 to expel blood into the left ventricle thereby eliminating stasis regions that result from enlarged atria from mitral regurgitation and/or atrial fibrillation, and improving cardiac output by increasing the pumping efficiency of the heart. It should be noted that both of these factors (mitral regurgitation resulting from valvular dysfunction or disease and atrial fibrilliation are known instigators and propogators of congestive heart disease. As the ventricles contract during systole, the atrium or atria are induced to expand allowing them to accept blood which can be pumped into the ventricles during the next cycle.

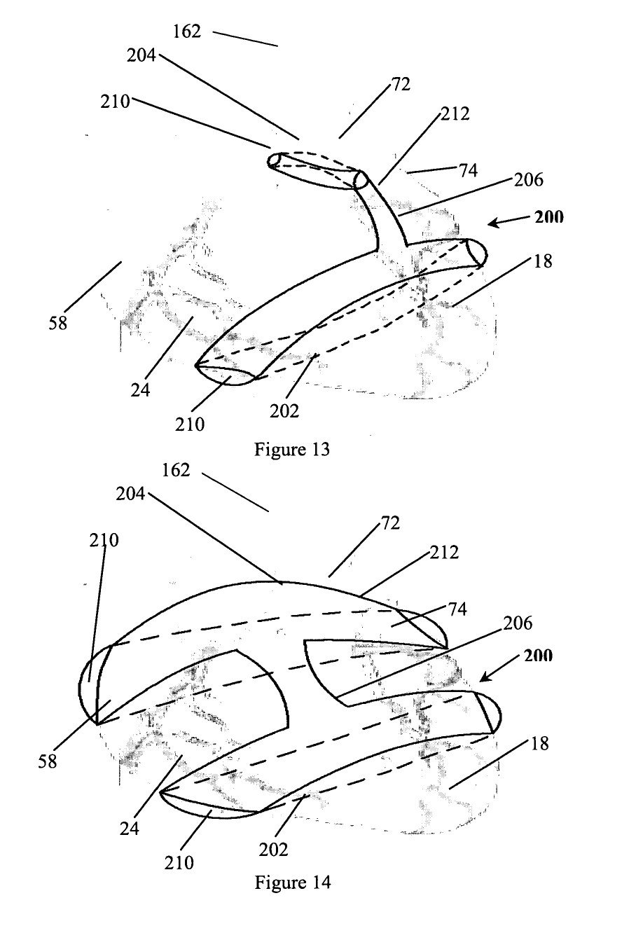

FIG. 13 shows the force transfer structure embodiments in FIGS. 6A to 6G and FIG. 7 secured to the heart with the source loop 202 about the ventricles 18 and 24 and the recipient loop 204 extending around the pulmonary artery 72. Again, expansion of the left and right ventricles 18 and 24 during diastole causes contraction of the pulmonary artery 72 thereby augmenting the pumping of blood into or towards the lungs. Contraction of the left and right ventricles 18 and 24 during systole causes the pulmonary artery 72 to expand to accept blood from the right ventricle.

FIG. 14 shows the force transfer structure with the recipient loop 204 covering the atria, the aorta, and the pulmonary artery to induce pulsatile motion in all three anatomic structures simultaneously in response to motion of the ventricles. As the ventricles expand during diastole, the aorta, pulmonary artery, and atria are simultaneously compressed thereby augmenting the pumping action and systemic blood flow. As the ventricles contract during systole, the aorta, pulmonary artery, and atria are allowed to or urged to expand thereby accepting and storing blood in preparation for the next stage in the cardiac cycle.

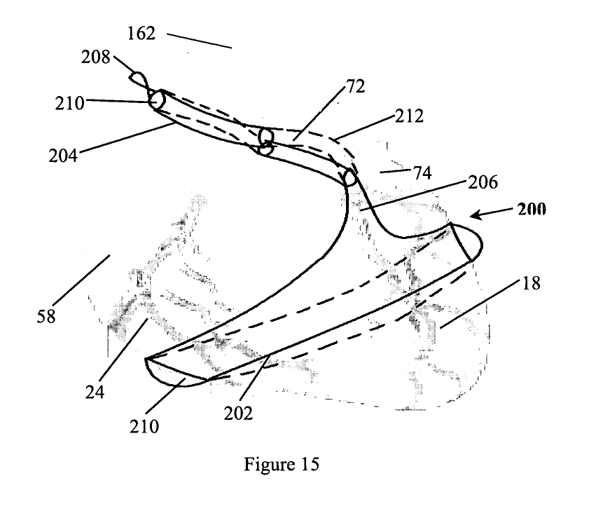

FIG. 15 shows a force transfer structure with the recipient loop 204 winding around the pulmonary artery 72 and aorta 162 to induce motion in these two anatomic structures in response to motion in the left and right ventricles 18 and 24.

Structure Materials and General Fabrication Methods

The embodiments of the entire invention described herein can be fabricated from various biological, metallic, and polymeric materials. For the force transfer structure embodiments incorporating fluid transmission capabilities, the structure can be fabricated from urethane, silicone, PET, polyester, nylon, polyurethane, expanded PTFE, FEP, composites of such materials, or a combination of materials. These force transfer structure embodiments can be fabricated by injection molding, extruding, blow molding, thermally forming, or otherwise producing the desired geometry of chambers interconnected together with lumens capable of transporting fluid from one chamber to another. Additional features such as a mesh or braid of tensile members or stiffening rods can be incorporated in the structure to impart stiffness, producing a resting geometry for the chambers, or further reinforce the heart. Alternatively, the force transfer structure can consist of a vessel, such as a saphenous vein, that has both ends tied and fluid inserted into the vessel, pericardium that is wound into a tube and secured in that shape with the ends closed, submucosal tissue formed into a closed tube, or other anatomic structure capable of defining chambers for fluid to be forcibly transferred coincident with the cardiac cycle.

For self-expanding components of the embodiments (e.g. some tensile member embodiments), those components are preferably fabricated from a superelastic, shape memory material like nitinol (nickel titanium alloy). These types of materials elastically deform upon exposure to an external force and return to their preformed shape upon reduction or removal of the external force. Superelastic shape memory alloys enable straining of the material numerous times without plastic deformation. The repetitive strain capability facilitates a limited systolic stretch to enable adequate cardiac output while limiting or restricting the possibility of over stretch and continuation of the cyclic damage.

Various components of the tensioning structures can be fabricated from shape memory alloys (e.g. nickel titanium) demonstrating stress-induced martensite at ambient temperature. Other shape memory alloys can be used and the superelastic material can alternatively exhibit austenite properties at ambient temperature. The composition of the shape memory alloy is preferably chosen to produce the finish and start martensite transformation temperatures (Mf and Ms) and the start and finish austenite transformation temperatures (As and Af) depending on the desired material response. When fabricating shape memory alloys that exhibit stress induced martensite the material composition is chosen such that the maximum temperature that the material exhibits stress-induced martensite properties (Md) is greater than Af and the range of temperatures between Af and Md covers the range of ambient temperatures to which the support members are exposed. When fabricating shape memory alloys that exhibit austenite properties and do not transform to martensite in response to stress, the material composition is chosen such that both Af and Md are less than the range of temperatures to which the supports are exposed. Of course, Af and Md can be chosen at any temperatures provided the shape memory alloy exhibits superelastic properties throughout the temperature range to which they are exposed. Nickel titanium having an atomic ratio of 51.2% Ni to 48.8% Ti exhibits an Af of approximately −20° C.; nickel titanium having an atomic ratio of 50% Ni to 50% Ti exhibits an Af of approximately 100° C. [Melzer A, Pelton A. Superelastic Shape-Memory Technology of Nitinol in Medicine. Min Invas Ther & Allied Technol. 2000: 9(2) 59-60].

Such superelastic components are able to withstand strain as high as 10% without plastically deforming. Materials other than superelastic shape memory alloys can replace superelastic materials in appropriate tensioning structure components provided they can be elastically deformed within the temperature, stress, and strain parameters required to maximize the elastic restoring force, thereby enabling the tensioning structures to exert a directional force in response to an induced deflection. Such materials include other shape memory alloys, bulk metallic glasses, amorphous Beryllium, suitable ceramic compositions, spring stainless steel 17-7, Elgiloy™, superelastic polymers, etc.

The tensile members of various force transfer structure embodiments can be fabricated from at least one rod, wire, suture, strand, strip, band, bar, tube, sheet, ribbon or other such raw material having the desired pattern, cross sectional profile, dimensions, or a combination of cross-sections. These raw materials can be formed from various standard means including but not limited to: extrusion, injection molding, press-forging, rotary forging, bar rolling, sheet rolling, cold drawing, cold rolling, using multiple cold working and annealing steps, or casting. When using superelastic materials or other alloys as the tensile members, they can be cut into the desired pattern and thermally formed into the desired three-dimensional geometric form. The tensile members can then be cut into the desired length, pattern or other geometric form using various means including, but not limited to, conventional abrasive sawing, water jet cutting, laser cutting, EDM machining, photochemical etching or other etching techniques. The addition of holes, slots, notches and other cut away areas on the support structure body facilitates the capability to tailor the stiffness of the implant.

The tensile members, especially those that employ the use of tubular or wire raw materials, can also be further modified via centerless grinding means to enable tensile members that are tapered (i.e. have a cross-sectional diameter on the proximal end of the structure that progressively ramps down to a smaller cross-section on the opposite or distal end).

When fabricating superelastic tensile members from tubing, the raw material can have an oval, circular, rectangular, square, trapezoidal, or other cross-sectional geometry capable of being cut into the desired pattern. After cutting the desired pattern, the tensile members are formed into the desired shape, heated, for example, between 300° C. and 600° C., and allowed to cool in the preformed geometry to set the shape of the tensile members.

When fabricating superelastic tensile members from flat sheets of raw material, the raw material can be configured with at least one width, W, and at least one wall thickness, T, throughout the raw material. As such, the raw sheet material can have a consistent wall thickness, a tapered thickness, or sections of varying thickness. The raw material is then cut into the desired pattern, and thermally shaped into the desired three-dimensional geometry. Opposite ends or intersections of thermally formed tensile members can be secured by using shrink tubing, applying adhesives, welding, soldering, mechanically engaging, utilizing another bonding means or a combination of these bonding methods. Opposite ends of the thermally formed tensile members can alternatively be free-floating to permit increased flexibility.

Once superelastic tensile members are fabricated and formed into the desired three-dimensional geometry, the supports can be electropolished, tumbled, sand blasted, chemically etched, ground, or otherwise treated to remove any edges and/or produce a smooth surface.

The previous discussions provide description of minimally invasive, force transmission structures used to treat degenerative heart disease in patients suffering any stage of congestive heart failure. In addition, the described inventions provide methods and devices to provide restriction of continued enlargement of the heart, potentially progressively reducing heart size via reverse remodeling (i.e. application of compressive force during both systole and diastole), improving atrial pump synchrony and efficienty thereby mitigating the morbidity effects of atrial fibrillation and finally decreasing valvular regurgitation associated with said enlargement.

It will be obvious to those skilled in the art that the structures described herein can be applied across a broad spectrum of organ structures to provide reinforcement and to limit enlargement facilitated by compensatory physiologic mechanisms.

### Claims

Though the invention has been described in reference to certain examples, optionally incorporating various features, the invention is not to be limited to the set-ups described. The invention is not limited to the uses noted or by way of the exemplary description provided herein. Numerous modifications and/or additions to the above-described embodiments would be readily apparent to one skilled in the art; it is intended that the scope of the present inventions extend to all such modifications and/or additions. It is to be understood that the breadth of the present invention is to be limited only by the literal or equitable scope of the following claims. That being said, we claim:

1. An apparatus to provide improved blood circulation in a mammal, the apparatus comprising:
a first portion adapted to interact with the left ventricle of a heart, and
a second portion adapted to interact with a blood bearing structure, said second portion being operatively connected with said first portion so that said second portion will cause contraction of said blood bearing structure to assist in driving blood circulation.

2. The apparatus of claim 1, wherein said second portion is adapted to at least partially surround an the aorta of the circulatory system.

3. The apparatus of claim 1, wherein said first and second portions are tensile members.

4. The apparatus of claim 1, wherein said first and second portions are fluid filled loops in open to each other.

5. An apparatus to provide improved blood circulation in a mammal, said apparatus comprising:
first and second means for transferring force between at least a portion of the heart and the aorta, the pulmonary artery or an atrium of the heart.

6. The apparatus of claim 5, wherein said first means comprises an encircling member adapted to at least partially surround a portion of the left ventricle of a heart.

7. The apparatus of claim 5, wherein said first means comprises an pair of members adapted to be attached to portions of the left ventricle of a heart.

8. The apparatus of claim 5, wherein said first means comprises a fluid-filled loop adapted to surround at least a portion of the left ventricle of a heart.

9. The apparatus of claim 5, wherein said second means comprises an encircling member adapted to at least partially surround at least a portion of the aorta, the pulmonary artery or an atrium of the heart.

10. The apparatus of claim 5, wherein said second means comprises a fluid-filled loop adapted to surround at least a portion of the aorta, the pulmonary artery or an atrium of the heart.

11. A method of providing for improved blood circulation in a mammal, the method comprising:
providing an apparatus as in any of claims 1-9, and
positioning said apparatus about at least a portion of the heart and the aorta, the pulmonary artery or an atrium of the heart.

12. A method of improving blood circulation in a mammal, with an apparatus placed about at least a portion of the heart and the aorta, the pulmonary artery or an atrium of the heart, the method comprising:
actuating said portion of the heart, and
transferring force from said portion of the heart to the aorta, the pulmonary artery or an atrium of the heart,
thereby causing contraction of the same to assist in driving blood circulation.
